(19) United States
(12) Patent Application Publication
(10) Pub. No.: US 2025/0228561 A1
(43) Pub. Date: Jul. 17, 2025

(54) SKIN TENSION-REDUCING SCAR-PREVENTING PLASTER

(71) Applicant: Beijing Easeng Medical Science Co., Ltd., Beijing (CN)

(72) Inventors: Wenya Wang, Beijing (CN); Cheng Peng, Beijing (CN); Xiang Zhao, Beijing (CN); Chaoqun You, Beijing (CN); Linfei Cheng, Beijing (CN); Peng Lu, Beijing (CN); Pan Gao, Beijing (CN); Long Yang, Beijing (CN)

(21) Appl. No.: 18/899,556

(22) Filed: Sep. 27, 2024

(30) Foreign Application Priority Data
Jan. 16, 2024 (CN) 202420104648.2

(51) Int. Cl. A61B 17/08; A61F 13/00; A61F 13/0246

(52) U.S. Cl. CPC A61B 17/085; A61F 13/025; A61B 2017/086; A61F 2013/00361

(57) ABSTRACT

The present disclosure provides a skin tension-reducing scar-preventing plaster, including a film stuck to the skin, where the film is divided into a left part, a silicone part and a right part, lower surfaces of the left part and the right part are both provided with adhesion gels, a lower surface of the silicone part is provided with a silicone gel, an area between the left part and the silicone part is a blank area, and an area between the right part and the silicone part is also a blank area; and a plurality of fixing blocks are mounted on the surface of the left part, a plurality of fixture blocks corresponding to the fixing blocks are mounted on the surface of the right part, traction strips are connected between the fixing blocks and the fixture blocks, and one end of the traction strips are connected with the fixing blocks.

### CROSS REFERENCE TO RELATED APPLICATION

This application is a Utility application which claims the benefit of and takes priority from Chinese Patent Application Serial No. 202420104648.2 filed on Jan. 16, 2024, from which priority is claimed under all applicable sections of Title 35 of the United States Code including, but not limited to, Sections 120, 363, and 365(c).

### TECHNICAL FIELD

The present disclosure belongs to the surgical technical field of closed wound, and particularly relates to a skin tension-reducing scar-preventing plaster.

### BACKGROUND

The scar is the appearance and histopathological change of normal skin tissues caused by various wounds, and an inevitable product during human wound repairing. When scar growth exceeds a certain limit, various complications such as appearance destroy and dysfunction will occur. For scar healing and resolution, wound closure and formation of new tissues are mainly from several days to several weeks of the injury in the initial healing stage, and the scar looks reddish, prominent or hard from the initial several weeks to several months after the wound closure; and it usually takes several months to one year even longer time, so the scar will become flat and soft gradually, the color will also fade gradually and be closer to the surrounding normal skin.

In the prior art, the patent No. CN213252506U discloses a novel external scar-removing plaster, including a plaster body, the plaster body includes an outer protective layer, an itching relieving layer, a treatment layer and an isolation box from outside to inside, and the outer protective layer, the itching relieving layer and the treatment layer are fixedly connected in respective; and the bottom of the treatment layer and the upper surface of the isolation box are adhered together through the stickiness of the treatment layer, a partition plate is fixedly connected into the isolation box and divides the isolation box into a first placement cavity and a second placement cavity.

It can be known from the prior art that preventing the generation of the skin scar is mainly based on two treatment ideas, firstly, the skin stretching in the scar area is reduced to inhibit the scar, as shown in the above-mentioned disclosed document, and secondly, the scar is reduced through the physical action of a silicone gel, its principle is that the structure of the scar tissue is changed by hydration and closure to the skin stratum corneum, thus allowing the scar to be softened and reduced and preventing the formation of new scar tissues; and the two treatment ideas select to use the corresponding treatment plan according to patient's needs and actual situations.

### SUMMARY

To address the technical issue proposed in the background art, the present disclosure provides a skin tension-reducing scar-preventing plaster.

To implement the above purpose, the present disclosure adopts the technical solution below:

A skin tension-reducing scar-preventing plaster, including a film stuck to the skin, where the film is divided into a left part, a silicone part and a right part, lower surfaces of the left part and the right part are both provided with adhesion gels, a lower surface of the silicone part is provided with a silicone gel, an area between the left part and the silicone part is a blank area, and an area between the right part and the silicone part is also a blank area; and
a plurality of fixing blocks are fixedly mounted on the surface of the left part, a plurality of fixture blocks corresponding to the fixing blocks are fixedly mounted on the surface of the right part, traction strips are connected between the fixing blocks and the fixture blocks, and one ends of the traction strips are fixedly connected with the fixing blocks while the other ends are clamped with the fixture blocks.

Preferably, a section of surfaces of the traction strips that are close to the fixture blocks are provided with clamping slots, clamping sheets are provided in the fixture blocks, the traction strips pass through the inside of the fixture blocks, and the clamping slots are clamped with the clamping sheets.

Preferably, the clamping slots are composed of a plurality of annular grooves with openings facing the fixing blocks, mounting openings are provided on inner walls of the fixture blocks, one ends of the clamping sheets that face the fixing blocks are fixedly mounted on inner walls of the mounting openings, and the clamping slots are clamped with the clamping sheets through the annular grooves.

Preferably, one ends of the traction strips that are away from the fixing blocks are fixedly connected with pull rings, and the pull rings are used for pulling the traction strips.

Preferably, upper and lower surfaces of the blank area on the film are smooth.

Preferably, the surface of the film is provided with release paper.

Compared with the prior art, the present disclosure has the following beneficial effects: in the present disclosure, the left part is close to the right part through traction, the film in the blank area shrinks first due to a relatively low physical strength, then the skin in the blank area is driven to shrink to the silicone part, such that the tension of the skin tissue in the scope of the silicone part is effectively reduced, thus playing a role in skin tension-reducing, helping the growth and healing of the skin tissue and further reducing the formation of the scar; and the cooperative use of the silicone and the tension-reducing structure plays a role in preventing the scar.

### BRIEF DESCRIPTION OF THE DRAWINGS

FIG. 1 is a schematic diagram of a solid structure of a skin tension-reducing scar-preventing plaster according to the present disclosure.

FIG. 2 is a schematic diagram of a plane structure of a skin tension-reducing scar-preventing plaster according to the present disclosure.

FIG. 3 is a schematic diagram of a front structure of a fixture block.

FIG. 4 is a schematic diagram of a side structure of a fixture block.

In the drawings: 1. Silicone part, 2. Film, 3. Left part, 4. Traction strip, 5. Clamping slot, 6. Fixing block, 7. Fixture block, 8. Mounting opening, 9. Pull ring, 10. Clamping sheet, 11. Blank area, 12. Right part.

### DETAILED DESCRIPTION OF THE EMBODIMENTS

The technical solutions in the embodiments of the present disclosure are clearly and completely elaborated below in combination with the drawings. It is apparent that the described embodiments are only a part of the embodiments of the present disclosure but not all.

In the description of the present disclosure, it is understood that orientation or position relationships indicated by the terms “upper”, “lower”, “front”, “rear”, “left”, “right”, “top”, “bottom”, “inner”, “outer” and the like are based on the orientation or position relationships as shown in the drawings, for ease of describing the present disclosure and simplifying the description only, rather than indicating or implying that the mentioned apparatus or element necessarily has a particular orientation and must be constructed and operated in the particular orientation. Therefore, these terms should not be understood as limitations to the present disclosure.

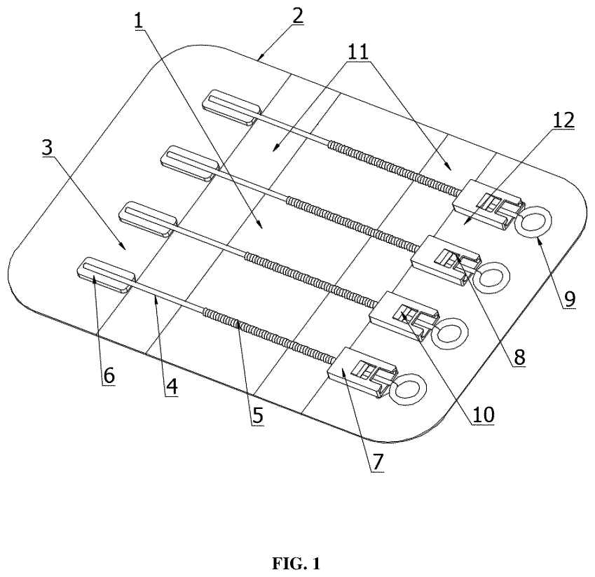

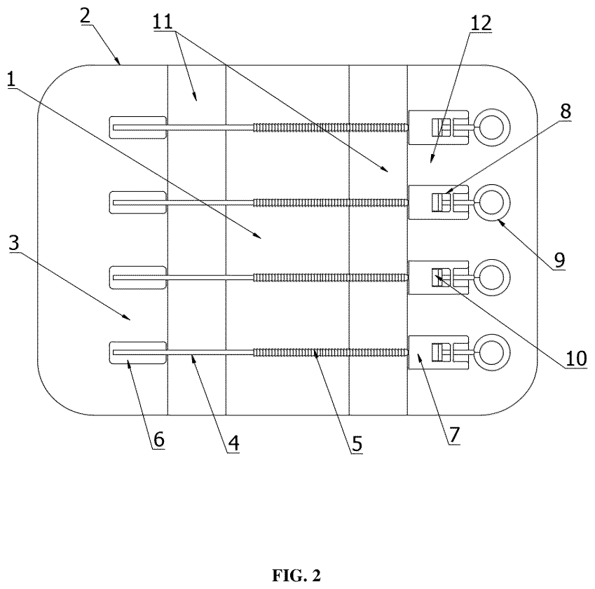

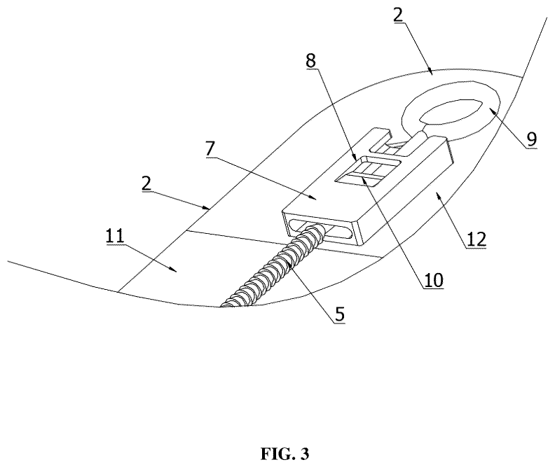

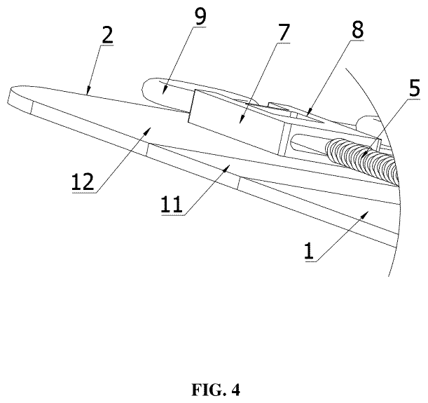

Referring to FIGS. 1-4, a skin tension-reducing scar-preventing plaster, including a film 2 stuck to the skin, where the film 2 is divided into a left part 3, a silicone part 1 and a right part 12, lower surfaces of the left part 3 and the right part 12 are both provided with adhesion gels, a lower surface of the silicone part 1 is provided with a silicone gel, an area between the left part 3 and the silicone part 1 is a blank area 11, and an area between the right part 12 and the silicone part 1 is also a blank area 11; and a plurality of fixing blocks 6 are fixedly mounted on the surface of the left part 3, a plurality of fixture blocks 7 corresponding to the fixing blocks 6 are fixedly mounted on the surface of the right part 12, traction strips 4 are connected between the fixing blocks 6 and the fixture blocks 7, and one ends of the traction strips 4 are fixedly connected with the fixing blocks 6 while the other ends are clamped with the fixture blocks 7.

One ends of the traction strips 4 that are away from the fixing blocks 6 are provided with pull rings 9, the pull rings 9 are used for pulling the traction strips 4, upper and lower surfaces of the blank area 11 on the film 2 are smooth, and the surface of the film 2 is provided with release paper (not shown in the drawings).

A section of surfaces of the traction strips 4 that are close to the fixture blocks 7 are provided with clamping slots 5, clamping sheets 10 are provided in the fixture blocks 7, the traction strips 4 pass through the inside of the fixture blocks 7, and the clamping slots 5 are clamped with the clamping sheets 10. When in use, users pull the traction strips 4 to be close to one ends of the fixture blocks 7, with the pulling, the clamping sheets 10 may be clamped at different positions of the clamping slots 5, thus narrowing the spacing between a second connecting sheet 12 and a first connecting sheet 3, allowing the skin to be reduced in tension and helping the skin recovery. The clamping slots 5 are composed of a plurality of annular grooves with openings facing the fixing blocks 6, mounting openings 8 are provided on inner walls of the fixture blocks 7, one ends of the clamping sheets 10 that face the fixing blocks 6 are fixedly mounted on inner walls of the mounting openings 8, the clamping slots 5 are clamped with the clamping sheets 10 through the annular grooves, and the openings of the annular grooves are arranged in opposite to the clamping sheets 10. In a process of pulling the traction strips 4, the elasticity of the skin tissue has a traction acting force on the fixing blocks 6, meanwhile the elasticity of the tissue also has the traction acting force on the fixture blocks 7, and the acting force on the fixing blocks 6 is opposite to that on the fixture blocks 7, just allowing the annular grooves to be clamped with the clamping sheets 10, thus implementing the fixing effect.

The silicone gel is the existing material, the silicone part 1 can form a layer of film on the wound to lock the moisture on the skin surface, this moisturizing environment helps to reduce the generation of the scar tissue, dryness may promote the formation of the scar tissue, and the silicone part 1 may maintain the temperature and pressure balance in the wound area, to help to reduce the growth of the scar tissue. Through the change of physical properties, the silicone plaster can reduce the hyperplasia of the scar tissue, thus reducing the tension in the scar area to some extent and reducing the possibility of stimulating the scar hyperplasia; the use of the silicone part 1 may reduce the redness and itching feeling of the scar, thus improving the appearance and feeling of the scar; and the silicone part 1 helps to adjust the arrangement of collagen fibers, making the scar smoother and softer.

When in use, the release paper on the lower surface of the film 2 is torn off, the release paper is used for keeping the viscosity of the gel, and the lower surface of the film 2 has the viscosity; first the silicone part 1 is pasted to the patient's scar, the scar-preventing plaster cannot be applied on the opening wound, the users pull the traction strips 4 by the pull rings 9 from one side of the second connecting sheet 12, with the pulling of the traction strips 4, the clamping sheets 10 may be clamped at different positions of the clamping slots 5, pulling will enable the first connecting sheet 3 to close to the second connecting sheet 12, and the tension of the skin tissue in the space position and the scope of the middle sheet 1 will be reduced effectively. In the pulling process, the using surface of the blank area 11 is compounded with nothing, so it is the softest part of the whole plaster and will shrink preferentially, the blank area 11 shows a shrinking state and is not pasted with the skin, so the skin will not shrink with the shrinking of the blank area, and the skin in the blank area will present a tension-reducing effect. In a process of skin healing, the two ends of the unhealed wound have no outward tension, so its new tissues may be pasted tightly for healing, allowing the deep layer of the skin at this part to be consistent with the surface layer in tension, helping the growing and healing of the skin tissue and further preventing the formation of the scar; and the cooperative use of the silicone and the tension-reducing structure plays a role in preventing the scar.

Reducing the skin tension vertical to the wound and away from the wound direction helps to relieve or prevent the formation of the scar, pressure reduction means that reducing the pressure at the wound can improve the blood circulation in this area when preventing the formation of the scar, and the pressure reduction can reduce the inflammatory reaction at the wound; the inflammation is a part of the normal healing process, but the excessive inflammatory reaction may lead to the hyperplasia of the scar tissue and also helps the collagen fibers to heal in a more nature arrangement way, thus reducing the formation of the hard scar and helping the scar tissue to integrate into the surrounding normal tissue better; and reducing the skin tension can reduce the cell stress level, thus reducing the release of chemical signals capable of promoting the formation of the scar. This device can play a more obvious pressure-reducing effect on a relatively great wound or a great-tension wound, such as the vicinity of a joint, chest/back, belly, hip, thigh, neck, high-pressure wound after cutting the enlarged tumor, etc.

In conclusion, the above are only the specific implementation modes of the present disclosure, but the scope of protection of the present disclosure is not limited to this. Those skilled in the art can make equal replacements or changes according to the technical solution and its concept of the present disclosure in the technical scope of the present disclosure, and those replacements or changes shall be covered by the scope of protection of the present disclosure.

What is claimed is:

1. A skin tension-reducing scar-preventing plaster, comprising a film (2) stuck to the skin, wherein the film (2) is divided into a left part (3), a silicone part (1) and a right part (12), lower surfaces of the left part (3) and the right part (12) are both provided with adhesion gels, a lower surface of the silicone part (1) is provided with a silicone gel, an area between the left part (3) and the silicone part (1) is a blank area (11), and an area between the right part (12) and the silicone part (1) is also a blank area; and
a plurality of fixing blocks (6) are fixedly mounted on the surface of the left part (3), a plurality of fixture blocks (7) corresponding to the fixing blocks (6) are fixedly mounted on the surface of the right part (12), traction strips (4) are connected between the fixing blocks (6) and the fixture blocks (7), and one ends of the traction strips (4) are fixedly connected with the fixing blocks (6) while the other ends are clamped with the fixture blocks (7).

2. The skin tension-reducing scar-preventing plaster according to claim 1, wherein a section of surfaces of the traction strips (4) that are close to the fixture blocks (7) are provided with clamping slots (5), clamping sheets (10) are provided in the fixture blocks (7), the traction strips (4) pass through the inside of the fixture blocks (7), and the clamping slots (5) are clamped with the clamping sheets (10).

3. The skin tension-reducing scar-preventing plaster according to claim 2, wherein the clamping slots (5) are composed of a plurality of annular grooves with openings facing the fixing blocks (6), mounting openings (8) are provided on inner walls of the fixture blocks (7), one ends of the clamping sheets (10) that face the fixing blocks (6) are fixedly mounted on inner walls of the mounting openings (8), and the clamping slots (5) are clamped with the clamping sheets (10) through the annular grooves.

4. The skin tension-reducing scar-preventing plaster according to claim 2, wherein one ends of the traction strips (4) that are away from the fixing blocks (6) are fixedly connected with pull rings (9), and the pull rings (9) are used for pulling the traction strips (4).

5. The skin tension-reducing scar-preventing plaster according to claim 1, wherein upper and lower surfaces of the blank area (11) on the film (2) are smooth.

6. The skin tension-reducing scar-preventing plaster according to claim 1, wherein the surface of the film (2) is provided with release paper.
